(19) United States
(12) Patent Application Publication
(10) Pub. No.: US 2026/0174762 A1
(43) Pub. Date: Jun. 25, 2026

(54) OLCEGEPANT FOR TREATING SEPSIS

(71) Applicant: Boehringer Ingelheim International GmbH, Ingelheim am Rhein (DE)

(72) Inventors: Thierry BOUYSSOU, Warthausen (DE); Paul NICKLIN, Biberach an der Riss (DE); Ines Vera Viktoria TRUEBENBACH, Biberach an der Riss (DE); Markus ZWICKL, Mittelbiberach (DE)

(21) Appl. No.: 19/128,437

(22) PCT Filed: Nov. 9, 2023
(86) PCT No.: PCT/EP2023/081243

§ 371 Date: May 8, 2025

(30) Foreign Application Priority Data
Nov. 11, 2022 (EP) 22206839.7

(51) Int. Cl. A61K 31/517; A61K 9/00; A61K 9/51; A61P 31/04

(52) U.S. Cl. CPC A61K 31/517; A61K 9/0019; A61K 9/5153; A61K 9/5161; A61P 31/04

(57) ABSTRACT

The present invention relates to the compound

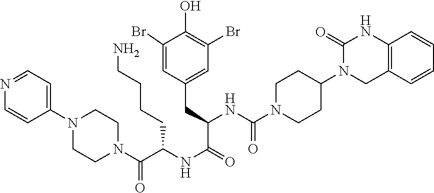 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

for use in a method for treating a patient with a disorder associated with bacterial or fungal severe sepsis, and bacterial or fungal septic shock and conditions arising therefrom, a pharmaceutical composition which contains said compound and its use as medicament for the treatment of bacterial or fungal severe sepsis and bacterial or fungal septic shock and conditions arising therefrom.

### FIELD OF THE INVENTION

The present invention relates to a compound for the use in the treatment of a patient with a systemic response to bacteria, fungi or circulating bacterial or fungal products and conditions arising therefrom, wherein the compound is

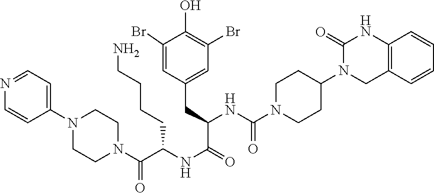 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

or a derivative thereof (olcegepant), and to a method of treatment of a patient with above mentioned systemic response, a pharmaceutical composition containing the compound and its use as medicament for the treatment of bacterial or fungal severe sepsis or bacterial or fungal septic shock and conditions arising therefrom.

### BACKGROUND OF THE INVENTION

In bacterial or fungal severe sepsis or bacterial or fungal septic shock an increase in vascular permeability is observed in several organs including but not limited to the lung, kidney, liver and heart. Interstitial fluid accumulation in these organs impairs their proper functioning (e. g. causing hypovolemia, hypotension, arrhythmia, glomerular filtration disruption, or impairment of the metabolism) and leads to organ failure followed by death. Regular antibiotics are not used for fungal infections because they are not effective. Sepsis, severe sepsis, and septic shock are disorders arising from the systemic inflammatory response to an infection (see Mitchell M. Levy et al., Crit Care Med. 2003 April; 31 (4): 1250-6). Sepsis is a disorder having both an infection (e.g., bacterial, fungal, abdominal trauma, gut perforation) and a systemic inflammatory response. This leads to increase in vascular permeability of several organs such as kidney, liver, heart and lung. Severe sepsis (sepsis with organ dysfunction) refers to sepsis with acute organ dysfunction caused by sepsis. Septic shock refers to persistent hypotension unexplained by other causes.

There is a need for compound formulations or dosage forms which can be used in a method for treating bacterial or fungal severe sepsis and bacterial or fungal septic shock. Unfortunately, no animal model mimics human sepsis. A number of anti-inflammatory drugs such as anti TNF-alpha or TLR4 inhibitors tested in pigs, monkeys or healthy volunteers challenged with LPS failed in septic patients.

Olcegepant, a calcitonin gene-related peptide (CGRP) receptor antagonist, is described in WO 98/11128 and by Doods et al. in the British Journal of Pharmacology 129:420-423, 2000.

Olcegepant given as a subcutaneous bolus injection in a mouse sepsis model only rescued 2 out of 10 mice (WO 2018/154015).

Messerer et al report that olcegepant given as an intravenous bolus injection in a pig sepsis model, did not show any survival improvement (British Journal of Anaesthesia 128 (5): 864-873, 2022).

Surprisingly, the present invention finds that a modified-release formulation of olcegepant improves the survival rate in a in a mouse model of polymicrobial septic shock.

### SUMMARY OF THE INVENTION

The present invention relates to the use of CGRP receptor antanonist olcegepant or a pharmaceutically acceptable salt thereof for the use in the treatment of a patient in which sepsis has been diagnosed, i.e. a patient with bacterial or fungal severe sepsis and bacterial or fungal septic shock and conditions arising therefrom, in particular conditions associated with bacterial or fungal parasites infections.

The invention relates to olcegepant or a pharmaceutically acceptable salt thereof, for use the treatment of a patient with bacterial or fungal sepsis, bacterial or fungal severe sepsis, or bacterial or fungal septic shock, or a condition arising therefrom which is selected from the group consisting of ARDS, related to infection, severe acute respiratory syndrome (SARS), middle eastern respiratory syndrome (MERS), Peritonitis and Puerperal (childbed) fever.

Furthermore, the invention relates to a method of treating a patient with bacterial or fungal sepsis, bacterial or fungal severe sepsis, or bacterial or fungal septic shock, or a condition arising therefrom which is selected from the group consisting of Acute Respiratory Distress Syndrome (ARDS), related to infection, severe acute respiratory syndrome (SARS), middle eatern respiratory syndrome (M ERS), Peritonitis and Puerperal (childbed) fever, comprising the administration of olcegepant or a pharmaceutically acceptable salt thereof.

In one embodiment the invention relates to a compound for use in a method for treatment of a patient with a systemic response to bacteria, fungi or circulating bacterial or fungal products and/or conditions arising therefrom, wherein the compound is the following CGRP receptor antagonist olcegepant or a pharmaceutically acceptable salt thereof:

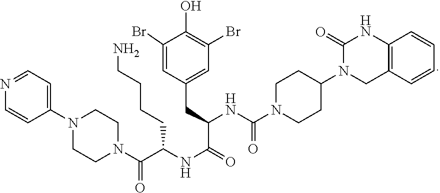 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

In a particular embodiment, the above invention relates to the compound olcegepant or a pharmaceutically acceptable salt thereof for use in the treatment of sepsis, wherein a sepsis patient has a CGRP plasma concentration in the range of 30 to 200 pmol/L (Arnalish et al al., Life Sci. 56:75-81, 1995, while plasma CGRP normal range should be lower than 10 pmol/L. In particular a patient's systemic response is characterized by interstitial fluid accumulation and/or hypotension.

Further, the above invention relates to a method of treating a patient with severe sepsis (i.e. sepsis with sepsis-induced organ dysfunction or tissue hypoperfusion (manifesting as hypotension, elevated lactate, or decreased urine output)) comprising the administration of olcegepant or a pharmaceutically acceptable salt thereof, wherein a sepsis patient has a CGRP plasma concentration in the range of 30 to 200 pmol/L (Arnalish et al al., Life Sci. 56:75-81, 1995, while plasma CGRP normal range should be lower than 10 pmol/L. In particular the patient's systemic response is characterized by interstitial fluid accumulation and/or hypotension.

A patient is diagnosed as having sepsis when symptoms such as fever, increased heart rate, low blood pressure, increased breathing, low urine output, severe pain and confusion are observed.

A further embodiment of the invention is a medicament prepared with the compound olcegepant or a salt thereof for use in the treatment of a patient with bacterial, viral or fungal sepsis.

Yet another embodiment of the invention is a pharmaceutical composition containing the compound olcegepant above or a pharmaceutically acceptable salt thereof together with a pharmaceutically acceptable carrier. Said composition is particularly for the use of olcegepant administered with a new method for the treatment of a patient with bacterial or fungal severe sepsis or bacterial or fungal septic shock.

Of particular interest is a pharmaceutical composition prepared as a modified, particularly a slow-release formulation of the CGRP receptor antagonist olcegepant or a pharmaceutically acceptable salt thereof, wherein said composition comprises 5-40%, particularly 5-20% of olcegepant or a pharmaceutically acceptable salt thereof

In one embodiment of the invention the CGRP receptor antagonist olcegepant can be prepared as a modified or slow release formulation, for example encapsulated in PLGA (poly(lactic-co-glycolic acid)) nanoparticles. Modified-release dosage delivers a drug with a delay after its administration or for a prolonged space of time. The FDA approved polymer PLGA is a biodegradable polymer composed of 75% lactic acid and 25% glycolic acid, forming nanospheres with a size lower than 200 nm and a negative surface charge, for the preparation of vaccines and drugs (Lim et al, Pharmaceutics 14:614, 2022, https://doi.org/10.3390/pharmaceutics14030614).

For the treatment of a patient such nanoparticles can be suspended in 0.1-0.4 w/v %, particularly 0.25 w/v % sodium carboxymethylcellulose (Na-CM C) and 0.01-0.02 v/v %, particularly 0.015 v/v % T ween 80 in a sterile 0.9% NaCl solution.

Olcegepant can be also delivered as modified or slow-release intravenous infusion at a rate such as 0.25 mg/2 ml/min for at least 8 hours with a physiological solution (such as 0.9% normal saline, or lactated ringer, or dextrose 5%, or human albumin (4%-5%) in saline or 6% hydroxyethyl starch (hespan) in order to keep the plasma concentration lower than 500 nmol/L in patients.

A further embodiment of the present invention is said pharmaceutical composition of the CGRP receptor antagonist olcegepant or a pharmaceutically acceptable salt thereof for use in the treatment of a patient with bacterial or fungal severe sepsis or bacterial or fungal septic shock comprises 5-40%, particularly 5-20% of olcegepant. The therapeutically effective amount of olcegepant is in the range from 0.1 to 90 wt.-% of the composition as a whole, preferably in the range from 0.5 to 50 wt.-% of the composition as a whole, or a pharmaceutically acceptable salt thereof.

### BRIEF DESCRIPTION OF THE SEVERAL VIEWS OF THE DRAWINGS

FIG. 1.1 shows the effect of olcegepant-PLGA in the mouse CLP model in Lung-dry tissue.

FIG. 1.2 shows the effect of olcegepant-PLGA in the mouse CLP model in Liver-dry tissue.

FIG. 1.3 shows the effect of olcegepant-PLGA in the mouse CLP model in Kidney-dry tissue.

FIG. 1.4 shows the effect of olcegepant-PLGA in the mouse CLP model in Heart-dry tissue.

FIG. 2.1 shows the effect of olcegepant-PLGA in the mouse Streptococcus pneumonia model on the BALF protein.

FIG. 2.2 shows the effect of olcegepant-PLGA in the mouse Streptococcus pneumonia model in the Lung-dry tissue.

FIG. 3.1 shows the effect of olcegepant-PLGA in the mouse lung ischemia/reperfusion model in the Lung-dry tissue.

FIG. 3.2 shows the effect of olcegepant-PLGA in the mouse lung ischemia/reperfusion model on the BALF protein.

FIG. 3.3 shows the effect of olcegepant-PLGA in the mouse lung ischemia/reperfusion model in the Heart-dry tissue.

FIG. 4.1 shows the effect of olcegepant-PLGA compared to olcegepant in saline on the survival rate in the mouse CLP model.

### DETAILED DESCRIPTION OF THE INVENTION

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Such salts can be acetates, ascorbates, benzenesulfonates, benzoates, besylates, bicarbonates, bitartrates, bromides/hydrobromides, edetates, camsylates, carbonates, chlorides/hydrochlorides, citrates, edisylates, ethane disulfonates, estolates esylates, formates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates, subacetates, succinates, sulfamides, sulfates, tannates, tartrates, teoclates, toluenesulfonates, triethiodides, trifluoroacetates, ammonium, benzathines, chloroprocaines, cholines, diethanolamines, ethylenediamines, meglumines and procaines.

Further pharmaceutically acceptable salts can be formed with cations from metals like aluminium, calcium, lithium, magnesium, potassium, sodium, zinc and the like (also see Pharmaceutical salts, Birge, S. M. et al., J. Pharm. Sci., (1977), 66, 1-19) or with cations with cations from ammonia, L-arginine, calcium, 2,2′-iminobisethanol, L-lysine, magnesium, N-methyl-D-glucamine, potassium, sodium and tris(hydroxymethyl)-aminomethane.

### Methods of Therapeutic Use

Olcegepant is effective for treating bacterial and fungal sepsis, severe sepsis and septic shock.

For use in the treatment of bacterial or fungal severe sepsis and bacterial or fungal septic shock olcegepant may be administered via a modified or slow-release formulation comprising olcegepant or a pharmaceutically acceptable salt thereof encapsulated in PLGA (poly [lactic-co-glycolic acid]). For treatment of a patient such nanoparticles can be suspended in 0.1-0.4%, particularly 0.25% sodium carboxymethylcellulose (Na-CM C) and 0.01-0.02%, particularly 0.015% Tween 80 in a sterile 0.9% NaCl solution.

Olcegepant can also be delivered as modified or slow release intravenous infusion at a rate such as 0.25 mg/2 ml/min for 8 hours with a physiological solution (such as 0.9% normal saline or lactated ringer, or dextrose 5%, or human albumin (4%-5%) in saline or 6% hydroxyethyl starch (hespan) in order to keep the plasma concentration lower than 500 nmol/L in patients.

Routes of administration include, but are not limited to, intravenously, intramuscularly, subcutaneously, intrasynovially, by infusion, sublingually, transdermally, orally, topically or by inhalation. The preferred modes of administration are oral and intravenous.

Olcegepant may be administered alone or in combination with adjuvants that enhance stability of the compound, facilitate administration of pharmaceutical compositions containing them in certain embodiments, provide increased dissolution or dispersion, increase inhibitory activity, provide adjunct therapy, and the like, including other active ingredients.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The literature reports a lack of efficacy of olcegepant when given as a bolus injection either intravenously or subcutaneously in animal models of sepsis:

Surprisingly, it was found that olcegepant given as a modified or slow-release formulation, significantly reduced vascular hyper-permeability and improved the survival rate in animal models of sepsis. Thus, olcegepant may be used for the treatment of bacterial or fungal severe sepsis.

### EXAMPLES

### CLP-Induced Polymicrobial Sepsis in the Mouse

Cecal ligation puncture (CLP) is a model of polymicrobial sepsis which consists of extruding fecal content in the abdominal cavity of the animals under anesthesia.

Two models were performed:
A cute model of CLP lasting 24 h to measure vascular hyper-permeability in lung, liver, kidney and heart tissue. The vascular hyper-permeability was followed by the extravasation of Evans blue injected intravenously and diffused and accumulated into the tissue. The vascular hyper-permeability was expressed as μg of Evans blue in 100 mg of dry tissue.
Chronic model of CLP lasting 8 days. CLP is performed on day 0 and the survival rate is followed for 8 consecutive days

### Acute Model of CLP: Vascular Hyper-Permeability

Mice were anesthetized with ketamine (80 mg kg⁻¹, i.p.) and xylazine (10 mg kg⁻¹, i.p.). A 1-1.5 cm abdominal midline incision was made, and the caecum was located and tightly ligated at half the distance between distal pole and the base of the caecum with 4-0 silk suture (mild grade). The caecum was punctured through-and-through once with a 21-gauge needle after medium ligation. A small amount of stool was extruded to ensure that the wounds were patent. Then the cecum was replaced in its original position within the abdomen, which was closed with sutures in layers. For negative control animals, a sham surgery was performed: sham-operated animals underwent identical laparotomy but without cecal ligation or puncture. The animals were resuscitated immediately after surgery with 1 mL subcutaneous normal saline and returned to their cages. Experiment was terminated at 24 hrs after CLP.

### Example 1: Effect of Olcegepant in PLGA on Acute Model of CLP

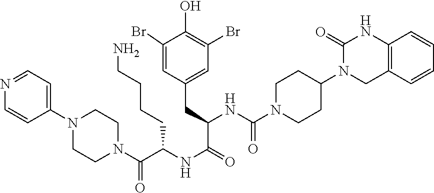 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

Olcegepant in PLGA formulation (128 mg olcegepant encapsulated in 512 mg PLGA nanoparticles ((Poly(D,L-lactide-co-glycolide) 75:25 called Resomer RG 752H manufactured by Evonik) corresponding to 640 mg in total (20% olcegepant) and suspended in 0.25% sodium carboxymethylcellulose ((Na-CM C) and 0.015% Tween 80 in sterile 0.9% NaCL solution or its vehicle (PLGA nanoparticles suspended in 0.25% sodium carboxymethylcellulose ((Na-CM C) and 0.015% Tween 80 in sterile 0.9% NaCL solution) was given subcutaneously (2 ml/kg) either prophylactically at 30 mg/kg 2 h before CLP or therapeutically at 10 or 30 mg/kg 2 h after CLP. 10 mice were included in each group.

Evans blue dye (0.1 mL at 40 mg/kg) was injected through each tail vein 30 minutes before euthanasia at 24 hours after CLP. Lung, kidney, liver and heart tissues are collected, and Evans blue was extracted in Formamide. The concentration of the Evans blue dye was calculated from a standard curve and expressed as μg/100 mg lung dry tissue. The data were analyzed by using commercially available software (Prism, version 8.3.0; GraphPad Software Inc., San Diego, CA). Different groups were compared with the one-way variance analysis (ANOVA) followed by the Dunnett test (e.g CLP group compared to treated groups). A II data were expressed as a mean±SEM. The limit of the significance was taken as p values less than 0.05 (p<0.05).

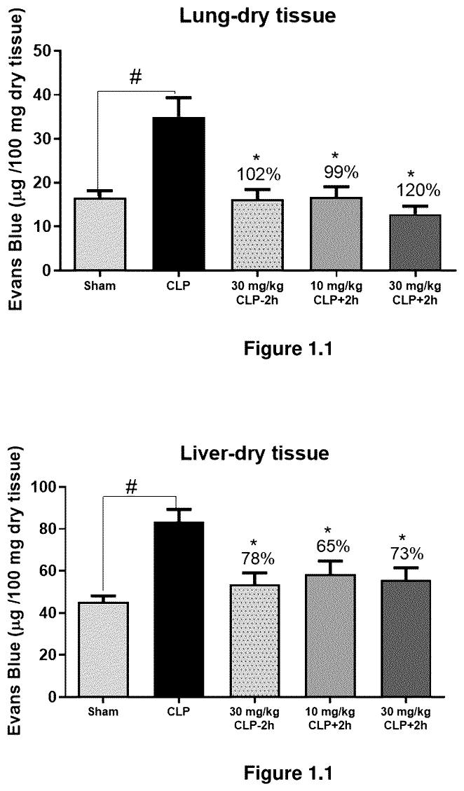

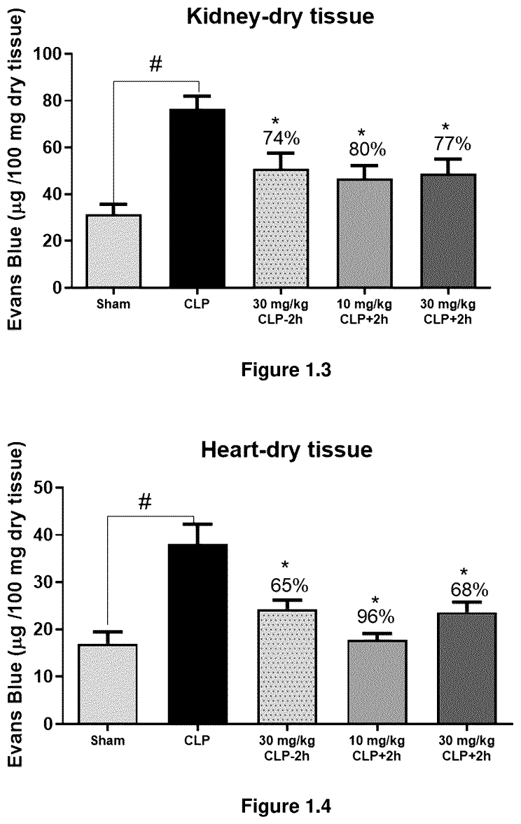

In the lung, Evans blue concentration (37 μg/100 mg dry tissue) in the CLP vehicle-treated group is significantly (p<0.05) higher than Evans blue in the sham group (15 μg/100 mg dry tissue) (FIG. 1.1). olcegepant in PLGA significantly (p<0.05) reduced Evans blue concentration either in prophylactic mode (102% inhibition at 30 mg/kg) or therapeutic mode (99% inhibition at 10 mg/kg and 120% at 30 mg/kg) (FIG. 1.1).

In the liver, Evans blue concentration (83 μg/100 mg dry tissue) in the CLP vehicle-treated group is significantly (p<0.05) higher than Evans blue in the sham group (45 μg/100 mg dry tissue) (FIG. 1.2). olcegepant in PLGA significantly (p<0.05) reduced Evans blue concentration either in prophylactic mode (78% inhibition at 30 mg/kg) or therapeutic mode (65% at 10 mg/kg and 73% at 30 mg/kg) (FIG. 1.2).

In the kidney, Evans blue concentration (80 μg/100 mg dry tissue) in the CLP vehicle-treated group is significantly (p<0.05) higher than Evans blue in the sham group (30 μg/100 mg dry tissue) (FIG. 1.3). olcegepant in PLGA significantly (p<0.05) reduced Evans blue concentration either in prophylactic mode (74% inhibition at 30 mg/kg) or therapeutic mode (80% inhibition at 10 mg/kg and 77% at 30 mg/kg) (FIG. 1.3).

In the heart, Evans blue concentration (39 μg/100 mg dry tissue) in the CLP vehicle-treated group is significantly (p<0.05) higher than Evans blue in the sham group (18 μg/100 mg dry tissue) (FIG. 1.4). olcegepant in PLGA significantly (p<0.05) reduced Evans blue concentration in prophylactic mode (65% inhibition at 30 mg/kg) and therapeutic mode (96% at 10 mg/kg and 68% at 30 mg/kg) (FIG. 1.4).

### Example 2: Effect of Olcegepant in PLGA on Streptococcus pneumonia-Induced Acute Lung Injury

Mice were inoculated intra-tracheally with Streptococcus pneumonia (10⁷ CFU/50 μL). Olcegepant in PLGA formulation (128 mg olcegepant encapsulated in 512 mg PLGA nanoparticles corresponding to 640 mg in total (20% olcegepant) and suspended in 0.25% sodium carboxymethylcellulose ((Na-CM C) and 0.015% Tween 80 in sterile 0.9% NaCL solution or its vehicle (PLGA nanoparticles suspended in 0.25% sodium carboxymethylcellulose ((Na-CM C) and 0.015% Tween 80 in sterile 0.9% NaCL solution) was given subcutaneously (2 ml/kg) therapeutically at 10 or 30 mg/kg, 2 h and 24 h after bacterial inoculation. The mice were euthanized 48 h after inoculation. 10 mice were included in each group. The lungs were flushed with 0.8 ml PBS in order to collect the broncho-alveolar-lavage (BALF) which was then centrifuged at 500 revolutions/min for 10 min and the supernatant was collected for the measurement of total protein according to Lowry measurement by absorbance at 660 nm.

Evans blue dye (0.1 mL at 40 mg/kg) was injected through each tail vein 30 minutes before euthanasia at 48 hours after bacterial inoculation. The lung tissues were collected, and Evans blue was extracted in Formamide. The concentration of the Evans blue dye was calculated from a standard curve and expressed as μg/100 mg lung dry tissue. The data were analyzed by using commercially available software (Prism, version 8.3.0; GraphPad Software Inc., San Diego, CA). Different groups were compared with the one-way variance analysis (ANOVA) followed by the Dunnett test (e.g Streptococcus pneumonia group compared to treated groups). All data were expressed as a mean±SEM. The limit of the significance was taken as p values less than 0.05 (p<0.05).

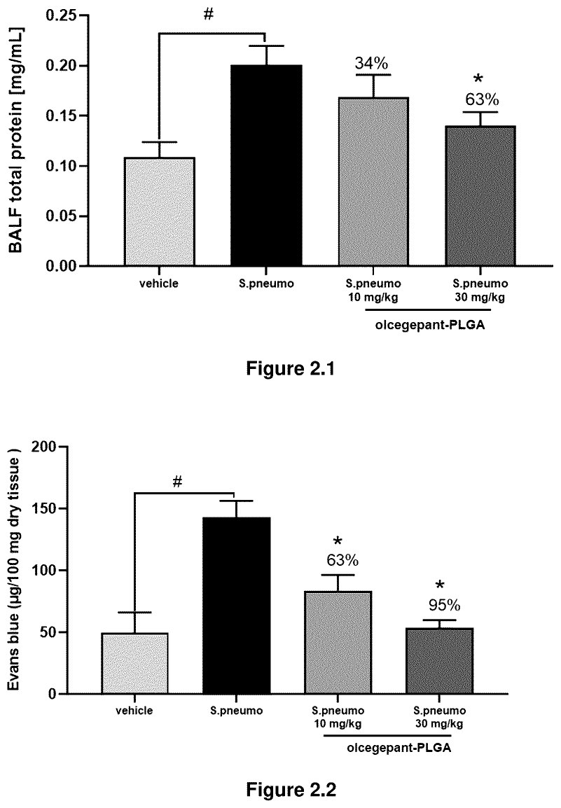

In the BAL F, Intra-tracheal inoculation of Streptococcus pneumonia induced lung edema is characterized by a significant accumulation of Broncho-Alveolar-Lavage protein (BALF protein). The origin of these proteins is albumin from the blood due to the vascular hyperpermeability and proteins from the membranes of lung alveolar cells, which are damaged. In the Streptococcus pneumonia groups, BALF protein (0.20 mg/ml BALF, FIG. 2.1) was significantly higher than BALF protein in the vehicle groups (0.10 mg/ml BALF, FIG. 2.1). olcegepant in PLGA given therapeutically significantly reduced BALF protein concentration of 63% at 30 mg/kg (FIG. 2.1).

In the lung, Evans blue concentration (150 μg/100 mg dry tissue) in the Streptococcus pneumonia group was significantly (p<0.05) higher than Evans blue in the sham group (50 μg/100 mg dry tissue) (FIG. 2.2). olcegepant in PLGA significantly (p<0.05) reduced Evans blue concentration in therapeutic mode (63% inhibition at 10 mg/kg and 95% at 30 mg/kg) (FIG. 2.2).

### Example 3: Effect of Olcegepant in PLGA on Lung Ischemia Reperfusion

Mice were anesthetized and mechanically ventilated. A left thoracotomy was performed and the pulmonary hilum of the left lung, including the bronchus, pulmonary artery and pulmonary vein, were occluded for 90 min with a clamp in order to cause pulmonary ischemia of the left lung. Upon removal of the clamp, the left lung was re-perfused and reventilated for 90 min. In time-matched sham-operated animals serving as controls, thoracotomy was performed without occlusion of the hilum.

Evans blue dye (0.1 mL at 40 mg/kg) was injected through each tail vein 30 minutes before the end of reperfusion.

At the end of the reperfusion period, bronchoalveolar lavage fluid (BALF) was performed with 3×0.5 mL PBS. Mice were perfused with PBS containing 5 mM EDTA. Left and right lungs and hearts were collected for assessment of Evans blue content. Evans blue dye (0.1 mL at 40 mg/kg) was injected through each tail vein 30 minutes before euthanasia. The lung and heart tissues were collected, and Evans blue was extracted in Formamide. The concentration of the Evans blue dye was calculated from a standard curve and expressed as μg/100 mg lung dry tissue. Olcegepant in PLGA formulation (128 mg olcegepant encapsulated in 512 mg PLGA nanoparticles corresponding to a formulation of 640 mg in total (20% olcegepant) and suspended in 0.25% sodium carboxymethylcellulose ((Na-CMC) and 0.015% Tween 80 in sterile 0.9% NaCL solution or its vehicle (PLGA nanoparticles suspended in 0.25% sodium carboxymethylcellulose ((Na-CMC) and 0.015% Tween 80 in sterile 0.9% NaCL solution) was given subcutaneously (2 ml/kg) prophylactically at 3, 10 or 30 mg/kg, 1 h prior to the ischemia.

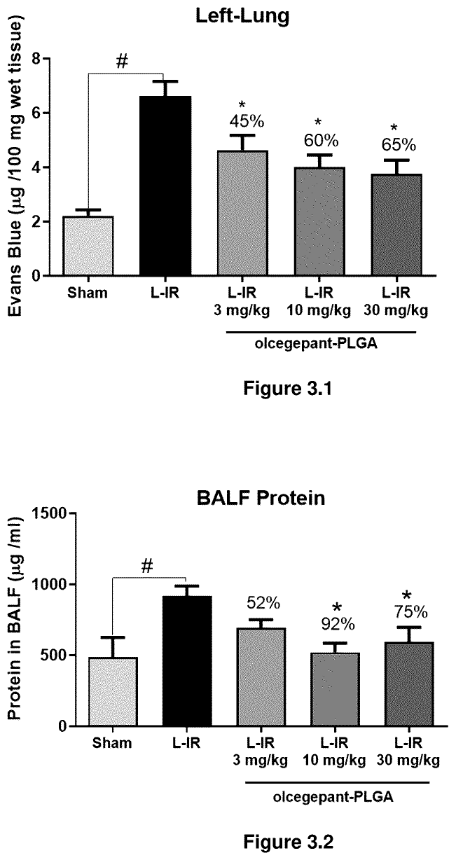

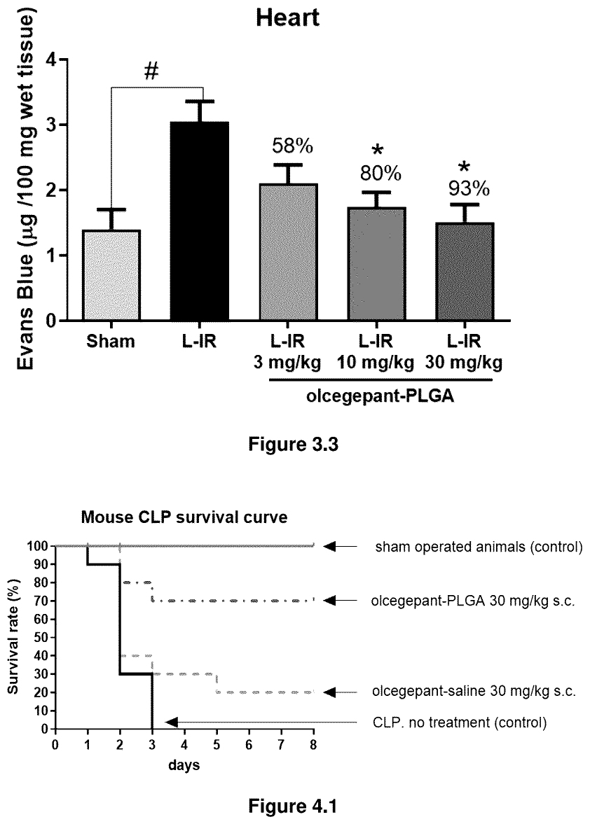

In the left lung, which was occluded, Evans blue concentration of 7 μg/100 mg dry tissue was significantly (p<0.05) higher than Evans blue in the sham group (2 μg/100 mg dry tissue) (FIG. 3.1). olcegepant in PLGA significantly (p<0.05) reduced Evans blue concentration in prophylactic mode (45% inhibition at 3 mg/kg, 60% at 10 mg/kg and 65% at 30 mg/kg) (FIG. 3.1).

In the BALF, Evans blue concentration of 1.8 μg/ml was significantly (p<0.05) higher than Evans blue in the sham group (0.4 μg/ml) (FIG. 3.2). olcegepant in PLGA significantly (p<0.05) reduced Evans blue concentration in prophylactic mode (54% inhibition at 10 mg/kg and 63% at 30 mg/kg) (FIG. 3.2).

In the heart, Evans blue concentration of 3.2 μg/100 mg dry tissue was significantly (p<0.05) higher than Evans blue in the sham group (1.5 μg/100 mg dry tissue) (FIG. 3.3). olcegepant in PLGA significantly (p<0.05) reduced Evans blue concentration in prophylactic mode (80% at 10 mg/kg and 93% at 30 mg/kg) (FIG. 3.3).

### Example 4: Chronic Model of CLP: Survival Rate

On day 0, mice were anesthetized with ketamine (80 mg kg⁻¹, i.p.) and xylazine (10 mg kg⁻¹, i.p.). A 1-1.5 cm abdominal midline incision was made, and the caecum was located and tightly ligated at half the distance between distal pole and the base of the caecum with 4-0 silk suture (mild grade). The caecum was punctured through-and-through once with a 21-gauge needle after medium ligation. A small amount of stool was extruded to ensure that the wounds were patent. Then the cecum was replaced in its original position within the abdomen, which was closed with sutures in layers. For negative control animals, a sham surgery was performed: sham-operated animals underwent identical laparotomy but without cecal ligation or puncture. The animals were resuscitated immediately after surgery with 1 mL subcutaneous normal saline and returned to their cages.

Olcegepant in PLGA formulation (128 mg olcegepant encapsulated in 512 mg PLGA nanoparticles corresponding to a formulation of 640 mg in total (20% olcegepant) and suspended in 0.25% sodium carboxymethylcellulose ((Na-CMC) and 0.015% Tween 80 in sterile 0.9% NaCL solution or its vehicle (PLGA microparticles suspended in 0.25% sodium carboxymethylcellulose ((Na-CM C) and 0.015% Tween 80 in sterile 0.9% NaCL solution) was given subcutaneously (2 ml/kg) therapeutically at 30 mg/kg, 2 h after CLP induction and once daily from day 1 to day 6.

Olcegepant solubilized in saline was also tested to compare its efficacy to the efficacy of olcegepant encapsulated in PLGA. Olcegepant in saline was given subcutaneously (2 ml/kg) therapeutically at 30 mg/kg, 2 h after CLP induction and once daily from day 1 to day 6.

10 mice were included in each group.

No mortality was observed in the sham group over 8 days, while all mice were found dead in the CLP group on day 3. Olcegepant in PLGA significantly reduced mortality at day 8 with a survival rate of 70% (7 mice out of 10 were still alive at day 8) (FIG. 4.1). Olcegepant solubilized in saline slightly reduced mortality at day 8 with a survival rate of 20% (2 mice out of 10 were still alive at day 8) (FIG. 4.1), which is in agreement with the data described in the patent WO 2018/154015, where olcegepant solubilized in saline and given at 100 mg/kg subcutaneous twice daily in the mouse CLP model, rescued 20% of the mice at day 4 (2 mice out of 10 were still alive at day 4).

1. A method for treating a patient with a systemic response to bacteria, fungi or circulating bacterial or fungal products and/or conditions arising therefrom, wherein the compound is the CGRP receptor antagonist 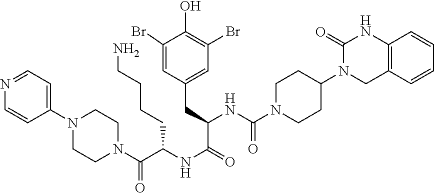 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]
or a pharmaceutically acceptable salt thereof.

2. The method of claim 1, wherein the patient's systemic response is characterized by interstitial fluid accumulation and/or hypotension.

3. The method of claim 1 wherein the patient has a CGRP plasma concentration of 30 to 200 pmol/L.

4. (canceled)

5. A pharmaceutical composition comprising olcegepant or a pharmaceutically acceptable salt thereof together with a pharmaceutically acceptable carrier.

6. The pharmaceutical composition of claim 5 comprising 5-40% of olcegepant or a pharmaceutically acceptable salt thereof.

7. The pharmaceutical composition of claim 6 comprising 5-20% of olcegepant or a pharmaceutically acceptable salt thereof.

8. The pharmaceutical composition of claim 7, further comprising 0.9% normal saline, or lactated ringer, or dextrose 5%, or human albumin (4%-5%) in saline or 6% hydroxyethyl starch (hespan).

9. The pharmaceutical composition of claim 7, wherein olcegepant is encapsulated in PLGA nanoparticles.

10. The pharmaceutical composition of claim 9 wherein the PLGA nanoparticles are suspended in 0.1-0.4% sodium carboxymethylcellulose (Na-CMC) and 0.01-0.02% Tween 80 in a sterile 0.8-1.0% NaCl solution.

11. The pharmaceutical composition of claim 9, wherein the PLGA nanoparticles are suspended in 0.25% sodium carboxymethylcellulose (Na-CMC) and 0.015% Tween 80 in a sterile 0.9% NaCl solution.

12. A method for treating a patient with bacterial or fungal severe sepsis or bacterial or fungal septic shock, comprising administering the pharmaceutical composition according to claim 5 to the patient.

13. A method for treating a patient with bacterial or fungal severe sepsis or bacterial or fungal septic shock having a CGRP plasma concentration of 30 to 200 pmol/L, comprising administering the pharmaceutical composition according to claim 5 to the patient, wherein
the pharmaceutical composition comprises a therapeutically effective amount of olcegepant or a pharmaceutically acceptable salt thereof in the range from 0.1 to 90 wt.-% of the composition as a whole, or in the range from 0.5 to 50 wt.-% of the composition as a whole.

14. The method according to claim 13, wherein the patient has a CGRP plasma concentration of 30 to 200 pmol/L compared to healthy conditions where CGRP plasma concentration is lower than 10 pmol/L.

15. A method for treating bacterial or fungal severe sepsis and bacterial or fungal septic shock, comprising administering a therapeutically effective amount of olcegepant to a patient in need thereof.
